(19) United States
(12) Patent Application Publication
(10) Pub. No.: US 2009/0264990 A1
(43) Pub. Date: Oct. 22, 2009

(54) Radiopaque Imprinted Ink Marker for Stent Graft

(75) Inventors: Walter Bruszewski, Guerneville, CA (US); Peggy Grills, Hidden Valley Lake, CA (US); Maria Acosta-Acevedo, Rohnert Park, CA (US); Masoumeh Mafi, Santa Rosa, CA (US); Tapan Mistry, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/106,403

(22) Filed: Apr. 21, 2008

(51) Int. Cl. A61F 2/06

(52) U.S. Cl. 623/1.34; 623/1.13

(57) ABSTRACT

A tubular synthetic endoluminal graft having at least one radiopaque ink marker pattern to radiographically delineate the surface of the graft cloth. The radiopaque ink marker includes a matrix of ink dots defining an annular band about a circumference of the graft. The endoluminal graft including at least one stent attached to the graft. The at least one stent may overlap or be positioned adjacent the radiopaque ink marker. The radiopaque ink marker may be utilized to facilitate creation of a fenestration in the side wall of the graft in situ to perfuse a side branch vessel.

### FIELD OF THE INVENTION

The present invention relates generally to a graft having radiopaque ink marker patterns imprinted thereon.

### BACKGROUND

Prostheses for implantation in blood vessels or other similar organs of the living body are, in general, well known in the medical art. For example, prosthetic vascular grafts constructed of biocompatible materials, such as Dacron or expanded, porous polytetrafluoroethylene (PTFE) tubing, have been employed to replace or bypass damaged or occluded natural blood vessels. In general, endovascular grafts typically include a graft anchoring component that operates to hold the tubular graft in its intended position within the blood vessel. Most commonly, the graft anchoring component is one or more radially compressible stents that are radially expanded in situ to anchor the tubular graft to the wall of a blood vessel or anatomical conduit. Thus, endovascular grafts are typically held in place by mechanical engagement and friction due to the opposition forces provided by the expandable stents.

In general, rather than performing an open surgical procedure to implant a bypass graft that may be traumatic and invasive, stent grafts are preferably deployed through a less invasive intraluminal delivery. More particularly, a lumen or vasculature is accessed percutaneously at a convenient and less traumatic entry point, and the stent graft is routed through the vasculature to the site where the prosthesis is to be deployed. Intraluminal deployment is typically effected using a delivery catheter with coaxial inner and outer tubes arranged for relative axial movement. For example, a self-expanding stent graft may be compressed and disposed within the distal end of an outer catheter tube distal of a stop fixed to the inner member. The catheter is then maneuvered, typically routed though a body lumen until the end of the catheter and the stent graft is positioned at the intended treatment site. The stop on the inner member is then held stationary while the outer tube of the delivery catheter is withdrawn. The inner member prevents the stent graft from being withdrawn with the sheath. As the sheath is withdrawn, the stent graft is released from the confines of the sheath and radially self-expands so that at least a portion of it contacts and substantially conforms with a portion of the surrounding interior of the lumen, e.g., the blood vessel wall or anatomical conduit.

Grafting procedures are also known for treating aneurysms. Aneurysms result from weak, thinned blood vessel walls that “balloon” or expand due to aging, disease and/or blood pressure in the vessel. Consequently, aneurysmal vessels have a potential to rupture, causing internal bleeding and potentially life threatening conditions. Grafts are often used to isolate aneurysms or other blood vessel abnormalities from normal blood pressure, reducing pressure on the weakened vessel wall and reducing the chance of vessel rupture. As such, a tubular endovascular graft may be placed within the aneurysmal blood vessel to create a new flow path and an artificial flow conduit through the aneurysm, thereby reducing if not nearly eliminating the exertion of blood pressure on the aneurysm.

While aneurysms can occur in any blood vessel, most occur in the aorta and peripheral arteries. Depending on the region of the aorta involved, the aneurysm may extend into areas of bifurcation or segments of the aorta from which smaller “branch” arteries extend. Various types of aortic aneurysms may be classified on the basis of the region of aneurysmic involvement. For example, thoracic aortic aneurysms include aneurysms present in the ascending thoracic aorta, the aortic arch, and branch arteries that emanate therefrom, such as subclavian arteries. Thoracoabdominal aortic aneurysm include aneurysms present in the descending thoracic aorta and branch arteries that emanate therefrom, such as thoracac intercostal arteries and/or the suprarenal abdominal aorta and branch arteries that emanate therefrom, such as renal, superior mesenteric, celiac and/or intercostal arteries. Lastly, abdominal aortic aneurysms include aneurysms present in the pararenal aorta and the branch arteries that emanate therefrom, such as the renal arteries.

Unfortunately, not all patients diagnosed with aortic aneurysms are presently considered to be candidates for endovascular grafting. This is largely due to the fact that most of the endovascular grafting systems of the prior art are not designed for use in regions of the aorta from which side branches extend. The deployment of endovascular grafts within regions of the aorta from which branch arteries extend present additional technical challenges because, in those cases, the endovascular graft must be designed, implanted, and maintained in a manner which does not impair the flow of blood into the branch arteries.

To accommodate side branches, a stent graft having a fenestration or opening in a side wall thereof is utilized. The fenestration is positioned to align with the ostium of the branch vessel after deployment of the stent graft. In use, the proximal end of the graft having one or more side openings is securely anchored in place, and the fenestrations or openings are configured and deployed to avoid blocking or restricting blood flow into the side branches. In some cases, another stent graft, often referred to as a branch graft, may then be deployed through the fenestration into the branch vessel to provide a path for blood flow to the branch vessel. One issue that exists in such a procedure is how to accurately position a fenestration in relation to the branch vessel. If the position of a fenestration is offset with respect to a branch vessel when the stent graft is deployed, it may be difficult to deploy guidewires and catheters from the stent graft into the branch vessel to enable correct positioning of the branch vessel stent graft, which in turn may result in the branch graft being deployed in such a manner that it kinks to such an extent that blood flow will not occur therethrough. Thus, there remains a need in the art for the development of new endovascular grafting systems and methods for providing perfusion to side branch vessels.

### SUMMARY OF THE INVENTION

A graft for implantation within a body lumen, includes a tubular body of a graft material and a radiopaque ink marker pattern imprinted on the graft material of the tubular body. The radiopaque ink marker pattern includes a matrix pattern of ink dots having a space between adjacent ink dots and the ink dots define an annular band around a circumference of the tubular body. In one embodiment, the graft may include a first stent attached to the tubular body and a second stent attached to the tubular body, wherein an unsupported body portion of graft material extends between the first support member and the second support member. The radiopaque ink marker pattern is imprinted on the graft material of the unsupported body portion of the graft.

Embodiments also relate to a method of creating a fenestration in a tubular graft in situ. A tubular graft is tracked to a target location within a body lumen, wherein the graft includes a first stent attached to the graft, a second stent attached to the graft, an unsupported body portion extending between the first support member and the second support member, and at least one radiopaque ink marker pattern imprinted on the unsupported body portion of the graft. The graft is positioned within the body lumen such that the radiopaque ink marker pattern is positioned in a known relationship with an ostium of a side branch vessel. The graft is radially expanded, and a puncture device is tracked to the radially expanded graft until a distal end of the puncture device is adjacent to the radiopaque ink marker pattern. A fenestration is created in the unsupported body portion of the graft to perfuse the side branch vessel.

### BRIEF DESCRIPTION OF DRAWINGS

The foregoing and other features and advantages of embodiments according to the present invention will be apparent from the following description as illustrated in the accompanying drawings. The accompanying drawings, which are incorporated herein and form a part of the specification, further serve to explain the principles of embodiments according to the present invention and to enable a person skilled in the pertinent art to make and use the invention. The drawings are not to scale.

FIG. 1 is an illustration of a portion of a tubular graft including a radiopaque ink marker pattern.

FIG. 2 is an illustration of the radiopaque ink marker pattern of FIG. 1 viewed under fluoroscopy.

FIG. 3 is an illustration of a portion of a tubular graft including a radiopaque ink marker pattern and a stent.

FIG. 4 is an illustration of the radiopaque ink marker pattern and stent of FIG. 3 viewed under fluoroscopy.

FIG. 5 is an illustration of a tubular graft including an unsupported portion having a radiopaque ink marker patterns thereon.

FIG. 6 is an illustration of a tubular graft including multiple unsupported portions each having a radiopaque ink marker patterns thereon.

FIG. 7 is an illustration of a stent graft delivery system.

FIGS. 8-10 illustrate a method of creating a fenestration in situ in a side wall of a tubular graft having an unsupported portion and a radiopaque ink marker patterns thereon.

### DETAILED DESCRIPTION

Specific embodiments are now described with reference to the figures, wherein like reference numbers indicate identical or functionally similar elements. The terms “distal” and “proximal” are used in the following description with respect to a position or direction of the delivery system relative to the treating clinician. “Distal” or “distally” are a position distant from or in a direction away from the clinician. “Proximal” and “proximally” are a position near or in a direction toward the clinician. While when referring to the stent graft or implant device, the term proximally refers to the end closest to the heart by way of blood flow path, while the term distal refers to the end away from the heart by way of blood flow path.

The following detailed description is merely exemplary in nature. Although the description herein is in the context of treatment of blood vessels such as the aortic, carotid, and renal arteries, embodiments according to the present invention may also be used in any other body passageways where deemed useful.

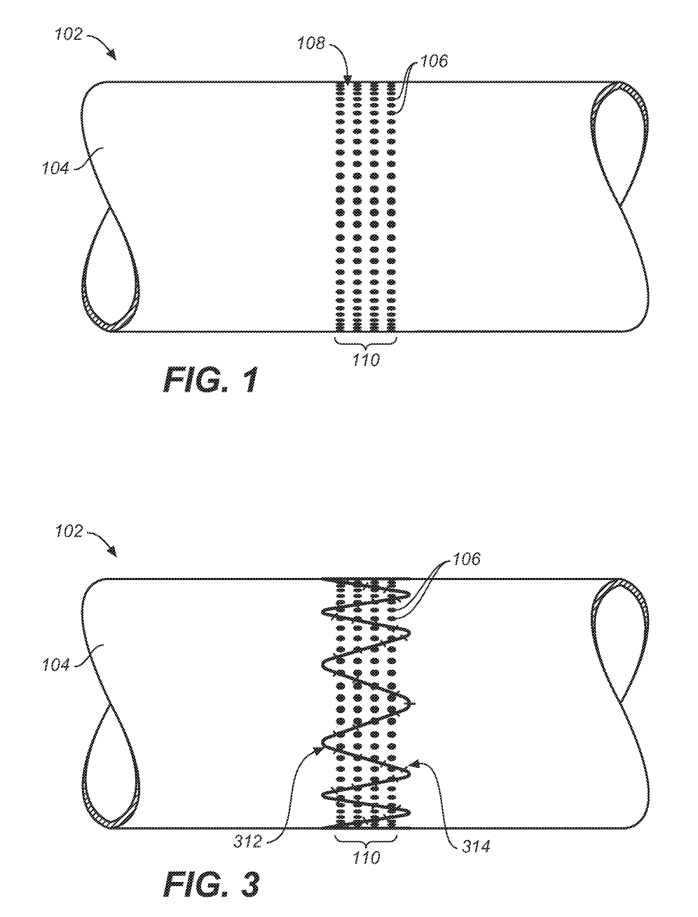

FIG. 1 is a side view of a portion of an endovascular graft 102 having a tubular body 104 and a radiopaque ink marker pattern 106 imprinted thereon. Graft 102 is a synthetic graft constructed from a suitable biocompatible material such as DACRON or other polyester fabric, or PTFE (polytetrafluoroethylene). The graft material is thin-walled so that graft 102 may be compressed into a small diameter, yet is capable of acting as a strong, leak-resistant fluid conduit when expanded to a cylindrical tubular form. Radiopaque ink marker pattern 106 includes a matrix or pattern of radiopaque ink dots 108 to radiographically delineate and provide fluoroscopic visualization of the location of portions of the surface of the graft cloth. Radiopaque ink marker pattern 106 includes a regular circumferential and longitudinal spacing between the individual ink dots 108. In one embodiment, the individual ink dots 108 have a diameter between 1-3 millimeters, and the spacing between the individual ink dots 108 is between 1-3 millimeters. The pattern of ink dots 108 of radiopaque ink marker pattern 106 defines a partial or continuous annular or circumferential band about a circumference of the tubular body 104 of graft 102. In an embodiment utilizing an annular band its width 110 is between 1-1.5 centimeters.

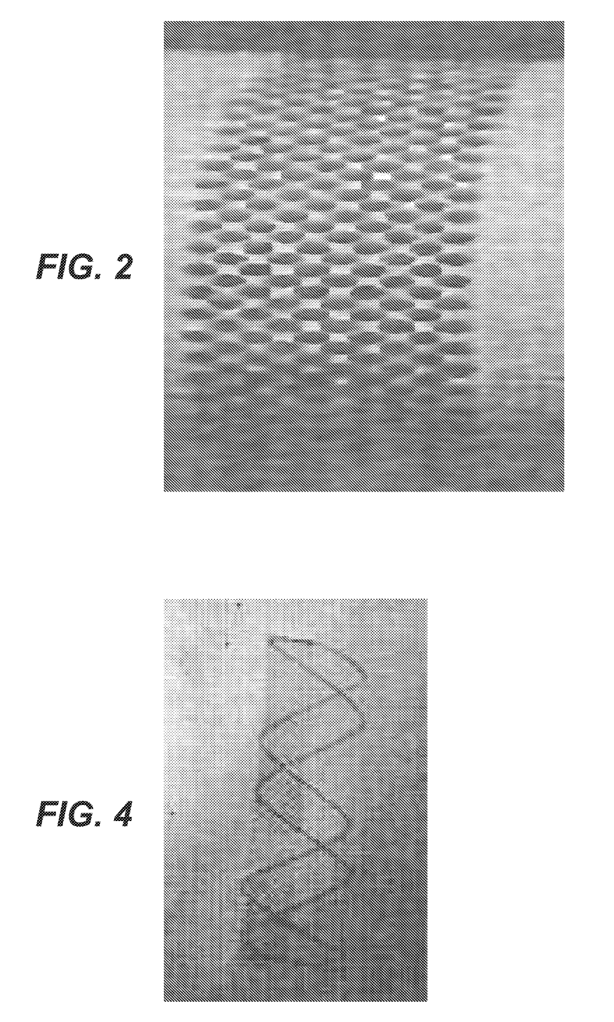

With reference to FIG. 2, it is demonstrated that radiopaque ink marker pattern 106 can be viewed under fluoroscopy by absorbing X-ray. Radiopaque ink marker pattern 106 is visible to an operator viewing, for example, an X-ray fluoroscopy device while deploying and/or positioning graft 102 into a target body vessel. Ink dots 108 of radiopaque ink markers 106 may be applied to graft 102 by a suitable imprinting technique, such as, for example, hand-painting, silk-screening, engraving, ink-jet, pad printing, or other type of imprinting technique. Radiopaque ink marker pattern 106 may be formed by any suitable radiopaque ink such as, for example, the ink produced by CI Medical, Inc. of Norton, Mass. The radiopaque ink is readily biocompatible and can be made to adhere to any surface, so that radiopaque ink dots 108 will not “fall off” or compromise the integrity of the graft. Utilization of radiopaque ink provides various advantages over conventional radiopaque markers that are typically sutured or otherwise attached to the surface of a graft. For example, radiopaque ink markers provide a packing density advantage in that the ink dots 108 take up less volume than conventional markers attached to the graft surface, thus allowing graft 102 to be packed into a delivery system with a reduced profile. In addition, conventional radiopaque markers are typically sutured to the surface of a graft and thus create suture holes that may lead to endoleak. Since radiopaque ink is imprinted on a surface of the graft, such suture holes are avoided. Also, conventional radiopaque markers attached to the surface of the graft require a gap between the end of the graft and the radiopaque marker to allow for an attachment mechanism such as sutures. Advantageously, ink dots 108 may be imprinted very close to the ends of the graft since no additional attachment means are required. Further, conventional radiopaque markers attached to the graft surface may displace or otherwise interfere with expandable support structures attached to the graft material. Since radiopaque ink marker pattern 106 is imprinted on a surface of the graft, such interference is avoided.

The annular band of width 110 defined by radiopaque ink marker pattern 106 is particularly advantageous because the annular band enables a 3D-like fluorographic visualization of graft 102. More particularly, a circumferential ring of the entire graft is visible as a circle or oval under fluoroscopy, whereas a conventional marker may only indicate a point on the graft surface. When a continuous cylindrical pattern of marker dots is applied the entire circumference of graft 102 may allow the operator to identify kinking or folding or deflection of the graft material, and also may permit the operator to identify if the graft is not completely open or deployed against the vessel wall. In an embodiment, radiopaque ink marker pattern 106 may be partially opaque. Controlling the opacity of radiopaque ink marker pattern 106 ensures that certain graft features, such as folds or kinks in the graft fabric, are not obscured by fully opaque markings, while still ensuring that radiopaque ink marker pattern 106 is viewable under fluoroscopic examination. Radiopaque ink marker pattern 106 will generally be applied as a radiopaque compound having radiopaque particles such as tungsten powder in a polyester matrix that is dissolved in a polyester solvent. The opacity may be controlled by regulating the proportion of radiopaque particles in the imprinting ink.

Referring now to FIGS. 3-4, the matrix or pattern of dots 108 defined by radiopaque ink marker pattern 106 is described in more detail. The matrix of dots 108 is a pattern that allows an expandable support structure to be attached to graft 102, while still providing the benefits of viewing the entire graft circumference, as described above. As shown in FIG. 3, a radially compressible annular support structure or stent 312 may be attached to a surface of graft 102. Stent 312 is preferably a self-expanding spring member that is deployed by release from a restraining mechanism, such as a sheath. For example, stent 312 may be constructed of a superelastic material, such as nitinol. Stents 312 may be attached or mechanically coupled to the graft material by stitching or suturing onto either the inside or outside of graft 102.

Stent 312 may have any suitable configuration. For example, stent 312 may be wavelike or sinusoidal patterned wire rings, a series of connected compressible diamond structures or other compressible spring members biased in a radially outward direction, which when released, bias the prosthesis into conforming fixed engagement with an interior surface of the vessel. Examples of such annular support structures are described, for example, in U.S. Pat. No. 5,713,917 and U.S. Pat. No. 5,824,041, which are incorporated by reference herein in their entirety. When used in an aneurysm exclusion device, the stents have sufficient radial spring force and flexibility to conformingly engage the prosthesis with the body lumen inner wall, to avoid excessive leakage, and prevent pressurization of the aneurysm, i.e., to provide a leak-resistant seal. Although some leakage of blood or other body fluid may occur into the aneurysm isolated by the graft prosthesis, an optimal seal will reduce the chances of aneurysm pressurization and resulting rupture.

When applied to a surface of graft 102, radiopaque ink marker pattern 106 will wick through and make a polymer bond with the graft material. It may be difficult to suture through the polymer bond, and thus stent 312 may not be attached overlapping or immediately adjacent to a continuous band of radiopaque ink around the circumference of the graft. However, the spacing within the matrix of dots 108 allows stent 312 to be attached overlapping or immediately adjacent to the annular band defined by radiopaque ink marker pattern 106 because sutures 314 may be placed through the graft material at the spacing within the matrix of dots 108. FIGS. 3-4 illustrate stent 312 overlapping with radiopaque ink marker pattern 106 on tubular body 104 of graft 102. In another embodiment, stent 312 may be positioned on tubular body 104 immediately adjacent the matrix of dots 108 that make-up radiopaque ink marker pattern 106. The regular of pattern marker dots on the surface of the tubular graft element, provides good visualization of the edge of the graft material, as several row or column are aligned to readily obstruct the passage of X-rays at the edge of the tube, i.e., the edge of the circular section, to create a dense line image in the X-ray at that point, while the dot pattern is diffuse and each dot is separately noticeable when viewed orthogonally.

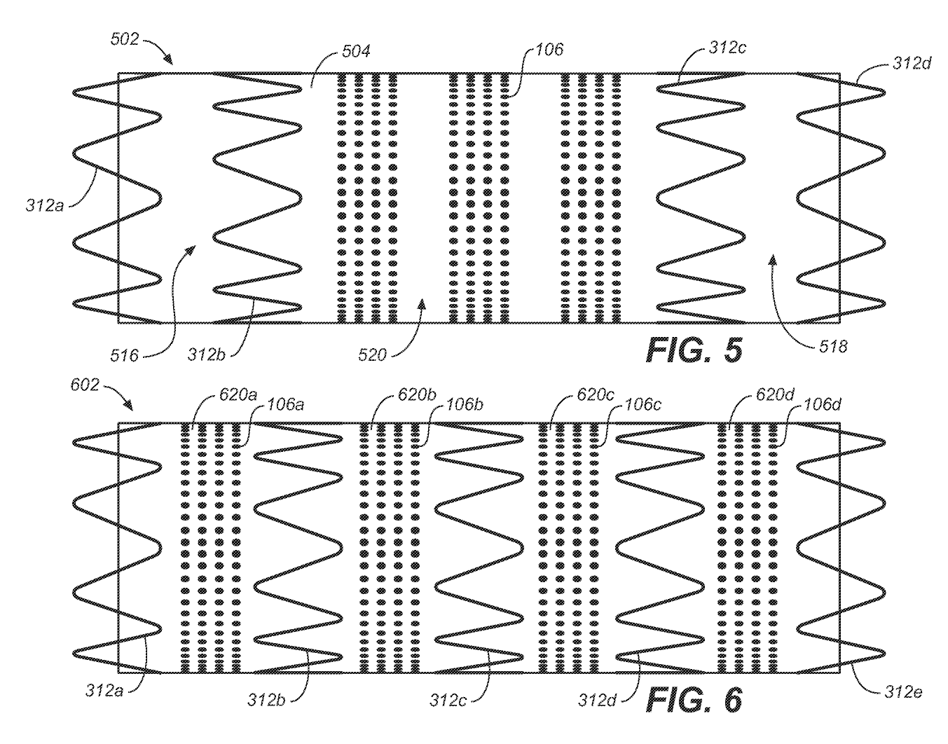

FIG. 5 is a side view of an endovascular graft 502 according to another embodiment. Graft 502 includes a synthetic graft material shaped as a tubular body 504 with a proximal supported portion 516, an intermediate unsupported body portion 520, and a distal supported portion 518. Proximal and distal supported portions 516, 518, respectively, include radially compressible stents attached thereto for supporting the ends of graft 502. FIG. 5 illustrates four stents 312a, 312b, 312c, 312d attached to graft 502; however, a greater or lesser number of stents may be utilized. Intermediate body portion 520 is solely graft material having no radial support along its length, i.e., is stent-free and unsupported, and extends between proximal and distal supported graft material portions 516, 518. Stents 312a-312d support the proximal and distal ends of graft 502 and/or bias the proximal and distal ends of graft 502 into conforming fixed engagement with an interior wall of a body lumen (not shown) while the unsupported body portion 520 is flexible permitting placement of the prosthesis in a highly curved anatomy, as well as reducing stresses on graft 502. The length of the unsupported body portion 520 may vary depending on the desired application.

Graft 502 having unsupported body portion 520 is particularly advantageous for use in a highly curved anatomy, such as the aortic arch. However, perfusion of side branch vessels extending from the aorta must be provided. In the embodiment of FIG. 5, three radiopaque ink marker patterns 106a, 106b, 106c are imprinted on unsupported body portion 520 of graft 102 to assist in creating in situ fenestrations in the graft; however, a greater or lesser number of radiopaque ink marker patterns may be utilized. Unlike conventional metal radiopaque markers attached to a graft surface, a puncture device may operate to create a fenestration in situ through graft material having radiopaque ink markers 106 imprinted thereon. An array of conventional metal radiopaque markers attached to the graft surface would interfere with the fenestration process. As will be explained in more detail herein, graft 502 is positioned and radially expanded within a target body lumen such that radiopaque ink marker pattern 106 is arranged in a known relationship with an ostium of a side branch vessel. A separate puncture device is tracked to the radially expanded graft until a distal end of the puncture device is adjacent to in a known relationship with radiopaque ink marker pattern 106, and a fenestration is created in unsupported body portion 520 of graft 502 to perfuse the side branch vessel. Radiopaque ink markers 106 allow the operator to assess the position of the puncture device because ink markers 106 show indentations and deflections in the graft material when the tip of the puncture device encounters the region to be fenestrated. In addition, the fenestration is created in unsupported body portion 520 of graft 502 and thus stents 312a-312d located at proximal and distal portions 516, 518 of graft 502 do not interfere with the puncture device. It is important to avoid close proximity of a puncture device to the stents because a fenestration immediately adjacent to a support member may result in damage to the support member and/or a fenestration with suboptimal robustness.

In the embodiment of FIG. 5, radially compressible stents 312a-312d are attached to both the proximal and distal portions 516, 518 of graft 502. In another embodiment, a radially compressible stent may be attached to only the proximal portion 516 of graft 502. Once the proximal portion 516 is expanded, blood flow enters and opens the remaining length of the graft. In yet another embodiment, a radially compressible annular support member may be attached to only the distal portion 518 of graft 502. Once the distal portion 518 is expanded, the remaining length of the graft partially expands such that blood flow enters and fully opens the remaining length of the graft.

Although FIG. 5 illustrates a single unsupported body portion located between the proximal and distal ends of a graft, a graft may include any number of unsupported body portions located between two stents. For example, FIG. 6 illustrates a graft 602 having multiple unsupported portions 620a, 620b, 620c, 620d and multiple stents 312a, 312b, 312c, 312d, 312e. Each unsupported portion is located between two stents and contains a radiopaque ink marker patterns 106a, 106b, 106c, 106d.

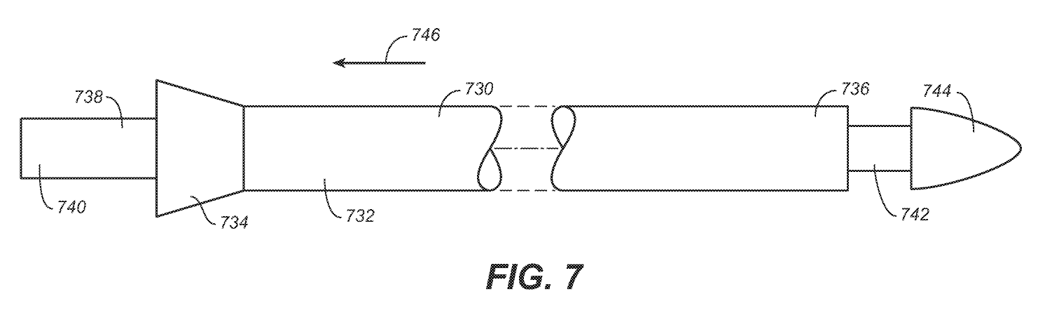

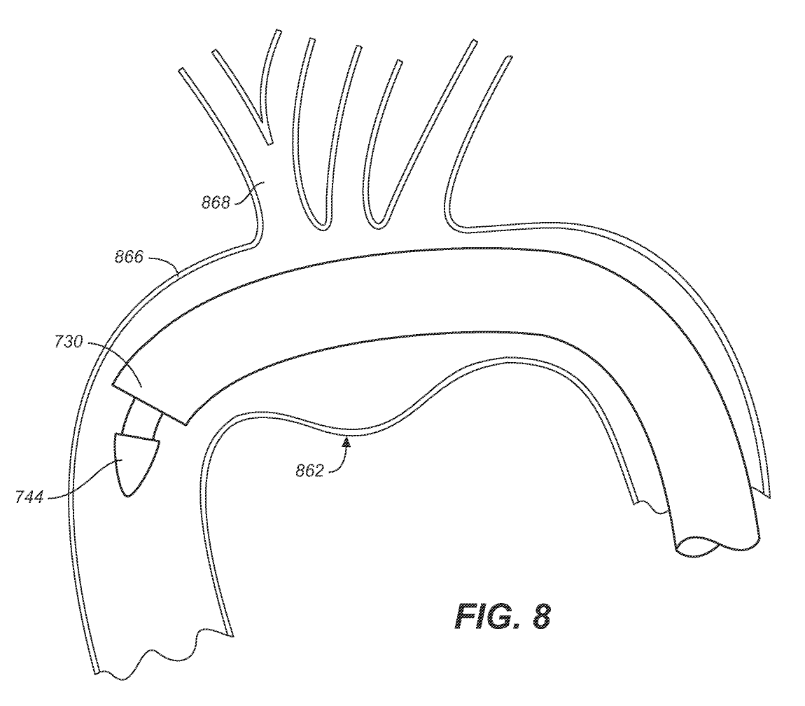

Grafts may be delivered by any suitable stent graft delivery system. For example, FIG. 7 illustrates a schematic side view of a graft delivery system for delivering and deploying a self-expanding stent graft. (A stent graft may also be balloon expandable.) The delivery system includes a retractable outer shaft 730 having a proximal end 732 and a distal end 736, and an inner shaft 738 having a proximal end 740 and a distal end 742. Outer shaft 730 defines a lumen extending therethrough (not shown), and inner shaft 738 slidably extends through the lumen of outer shaft 730 to a distal tip 744 of the graft delivery system. Distal tip 744 is coupled to distal end 742 of inner shaft 738, and may be tapered and flexible to provide trackability in tight and tortuous vessels. In an embodiment, inner shaft 738 may define a guidewire lumen (not shown) for receiving a guidewire (not shown) therethrough. When the guidewire lumen is present, inner shaft 738 may be advanced over an indwelling guidewire to track the delivery system to the target site. Alternatively, inner shaft 738 may instead be a solid rod (not shown) without a lumen extending there through. In an embodiment where inner shaft 738 is a solid rod, inner shaft 738 is tracked to the target site with the assistance of tapered distal tip 744.

Outer shaft 730 is provided to cover a graft (not shown in FIG. 7) mounted on the distal end 742 of inner shaft 738 while the graft delivery system is tracked through a body lumen to the deployment site. Outer shaft 730 is movable in an axial direction along and relative to inner shaft 738 and extends to a proximal portion of the graft delivery system where it may be controlled via an actuator, such as a handle 734 to selectively expand the graft mounted on distal end 742 of inner shaft 738. Outer shaft 730 in a non-retracted position contains the graft in a constrained diameter configuration. Handle 734 may be a push-pull actuator that is attached or connected to proximal end 732 of outer shaft 730. To expand the graft, while holding proximal end 740 of inner shaft 738 fixed, handle 744 is pulled in order to proximally retract outer shaft 730. Alternatively, the actuator may be a rotatable knob (not shown) that is attached or connected to proximal end 732 of outer shaft 730 such that when the knob is rotated, outer shaft 730 is retracted in a proximal direction to expand the graft. Thus, when the actuator is operated, i.e., manually turned or pulled, outer shaft 730 is proximally retracted over inner shaft 738 in a proximal direction as indicated by directional arrow 746. As illustrated in FIG. 7, outer shaft 730 is in a non-retracted, delivery configuration.

The graft may be mounted on distal end 742 of inner shaft 738 by any suitable configuration known in the art. For example, attachment bands extending between the graft and the inner shaft may be used for acting as a means for retaining the graft in place during delivery. The attachment bands eventually release the graft by self-expansion. Other means may be used for retaining the graft in place within graft delivery system 100 during delivery. For example, the graft may be held in frictional engagement with the graft delivery system by the inclusion of slots, ridges, pockets, or other prosthesis retaining features (not shown) formed into the exterior surface of the inner shaft to further ensure secure mounting of the graft as it is tracked transluminally to the target site. In addition, a cap may be coupled to the distal end of the inner shaft to retain the graft in a radially compressed configuration. An actuator at the proximal portion of the system may precisely control the release of the graft from the cap and from the radially compressed configuration. Such delivery systems may for example as described in U.S. Pat. No. 7,264,632 to Wright et al., which is hereby incorporated by reference in its entirety and the such similar delivery systems are well known in the art.

Referring now to FIGS. 8-10, a method of implanting a graft within an aneurysm 862 and creating a fenestration in a side wall of graft 502 in situ utilizing radiopaque ink marker patterns imprinted on a surface of the graft according to an embodiment hereof is described. FIG. 8 is a side view of a graft delivery system disposed within aortic arch 866. Aortic arch 866 has multiple side branch vessels 866 extending therefrom, including the left subclavian artery, the left common carotid artery, and the brachiocephalic artery, which further branches into the right subclavian artery and the right common carotid artery. The following method of creating a fenestration in a side wall of a graft in situ is described to provide perfusion to the brachiocephalic artery, but it will be understood that the method may be utilized for providing perfusion to the left subclavian artery or the left common carotid artery, as well as branch side vessels of other vessels beyond the aortic arch. The graft delivery system is tracked to and properly positioned within aortic arch 866 such that the graft to be delivered, such as graft 502, spans aneurysm 862. In use, the graft is preloaded into the delivery system with the stents held in a radially compressed configuration. Outer shaft 730 is placed over the graft to restrain the graft in the compressed configuration and prevent it from damaging or catching on the luminal wall as it is delivered to the aneurysm site. Methods and apparatus for delivering the graft intravascularly are generally known in the art and may be used to place the graft delivery system within the vasculature and deliver the graft to the deployment site. For example, the graft may be guided to the deployment site using fluoroscopic imaging.

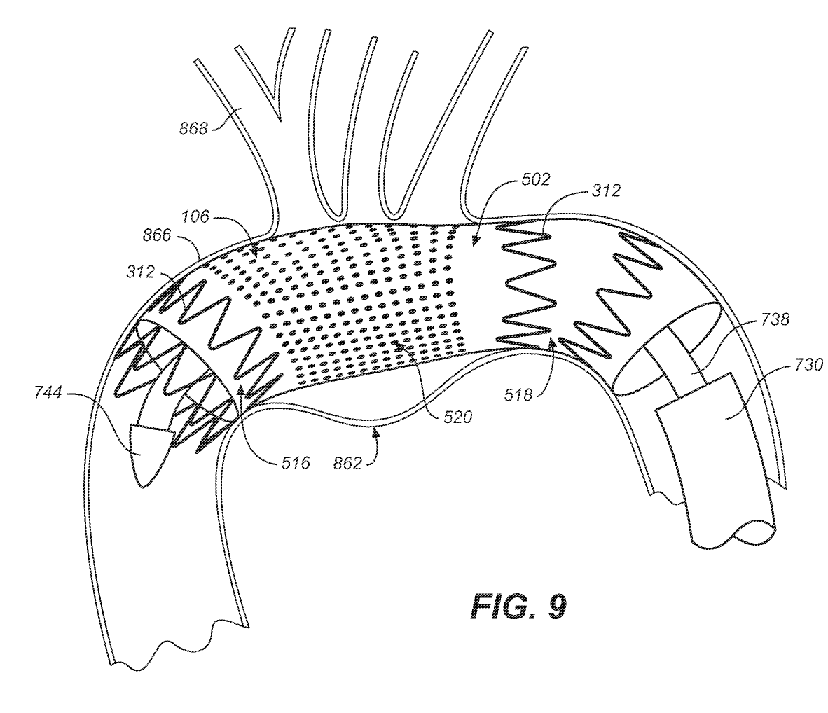

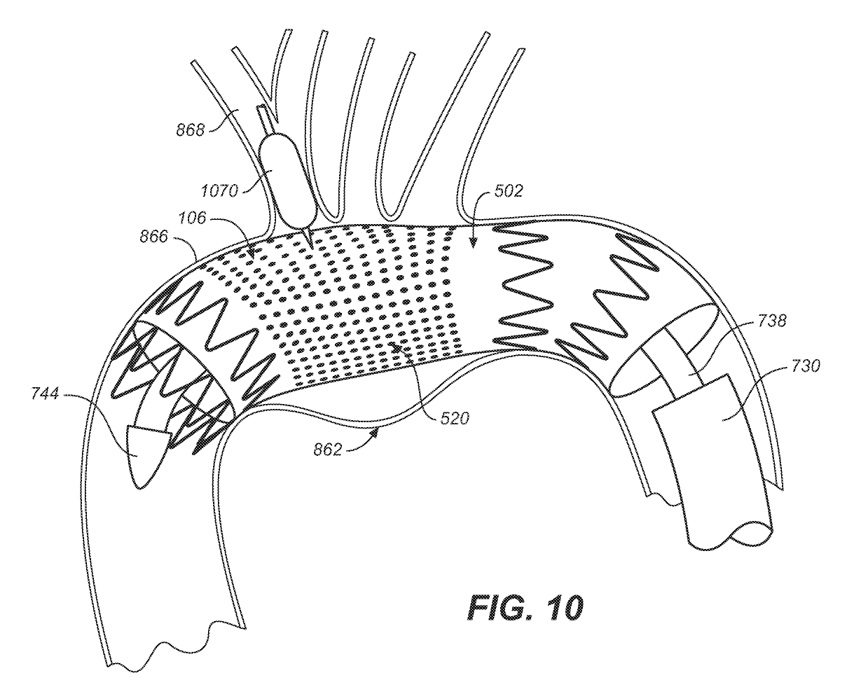

When a distal portion of the graft delivery system is located at the desired deployment site, outer shaft 730 is retracted from its position over the graft and the graft self-expands to engage the inner wall of the body lumen. As shown in FIG. 9, graft 502 is shown in deployed or expanded configuration. Stents 312 are provided at the proximal and distal portions 516, 518 of the graft for expanding, fixing, and sealing graft 502 to the vessel wall. In an alternate embodiment, where a stent is only located on one of a proximal end or a distal end of the graft, blood flow enters and opens the remaining length of the graft. Graft 502 includes unsupported or stent-free body portion 520, and a continuous plurality of radiopaque ink markers 106 imprinted on unsupported body portion 520 of the graft. Graft 502 is positioned within the body lumen such that radiopaque ink marker pattern 106 is positioned in a known relationship with an ostium of side branch vessel 868. Pressure and flow through the lumen of graft 502 stabilizes the surface of graft 502 and makes the graft material fenestratable as described below.

As shown in FIG. 10, a separate puncture device 1070 is delivered to create a fenestration in situ to perfuse side branch vessel 868 in the side of graft 502 for perfusion of a side branch vessel 868. Puncture device 1170 is delivered through the side branch vessel 868 in a retrograde fashion such that puncture device 1170 is delivered from an opposing side and initially encounters the outside surface of graft 502. Puncture device 1170 may be a dilator-needle combination device having a pointed tip sufficient for puncturing through the material of graft 502. Embodiments of the present structure may be used with any conventional puncture device capable of creating a fenestration in graft 502. Thus, it will be apparent to those of ordinary skill in the art that any features of the puncture device discussed herein are exemplary in nature. For example, the puncture device may be any puncture device known in the art, including but not limited to those shown or described in US Patent Application 2007-0208256 to Marilla, or as depicted in WO 2007/082343 or U.S. application Ser. No. 11/939106 filed on Nov. 13, 2007 and incorporated by reference herein in its entirety (lateral support for the graft during the puncture operation is not shown, but may be supplied by catheter based or stent graft structures providing sufficient lateral force needed to support the graft material during the puncture process). Puncture device 1070 is tracked to the radially expanded graft 502 until a distal end of puncture device 1070 is adjacent to radiopaque ink marker pattern 106. Using fluoroscopic imaging, indentations of the graft material with the distal end of puncture device 1170 is visible to the operator due to the continuous radiopaque ink marker pattern 106. Thus the radiopaque ink marker pattern 106 assure proper positioning, visualization, and usage of puncture device 1070 prior to creating a fenestration in unsupported body portion 520 of graft 502. Once puncture device 1070 is in place adjacent a receiving area of graft 502 where a fenestration is to be created, puncture device 1070 according to its operation punctures a side wall of graft 502 to perfuse side branch vessel 868.

If desired, puncture device 1070 may then moved to a second side branch vessel in need of perfusion, and the process is repeated to create additional fenestrations in a side wall of graft 502. Once fenestrations have been created in graft 502 as desired, puncture device 1070 is removed. The graft delivery system may be retracted and removed from the patient, while graft 502 remains expanded in the vessel against the vessel wall to provide an artificial lumen for the flow of blood.

While various embodiments have been described above, it should be understood that they have been presented by way of illustration and example only, and not limitation. It will be apparent to persons skilled in the relevant art that various changes in form and detail can be made therein without departing from the spirit and scope of the invention. It will also be understood that each feature of each embodiment discussed herein, and of each reference cited herein, can be used in combination with the features of any other embodiment. All patents and publications discussed herein are incorporated by reference herein in their entirety.

What is claimed is:

1. A graft for implantation within a body lumen, the graft comprising:
a tubular body of a graft material;
a radiopaque ink marker pattern imprinted on the graft material of the tubular body, wherein the radiopaque ink marker includes a matrix of ink dots having a space between adjacent ink dots and the ink dots define an annular band around a circumference of the tubular body.

2. The graft of claim 1, wherein the annular band has a width of between 1-1.5 centimeters.

3. The graft of claim 1, wherein the space between adjacent ink dots is between 1-3 millimeters.

4. The graft of claim 1, further comprising: at least one stent sutured to the tubular body.

5. The graft of claim 4, wherein the radiopaque ink marker is adjacent to the support member on the tubular body.

6. The graft of claim 4, wherein at least a portion of the support member overlaps with the matrix of ink dots of the radiopaque ink marker.

7. The graft of claim 1, wherein the radiopaque ink marker is partially opaque.

8. A graft for implantation within a body lumen, the graft comprising:
a graft material formed into a tubular body;
a first stent attached to the tubular body and a second stent attached to the tubular body, wherein an unsupported body portion of graft material extends between the first support member and the second support member; and
a radiopaque ink marker pattern imprinted on the graft material of the unsupported body portion of the graft.

9. The graft of claim 8, wherein the radiopaque ink marker defines an annular band around a circumference of the tubular body.

10. The graft of claim 9, wherein the first and second annular supports member are sutured to the tubular body.

11. The graft of claim 9, wherein the annular band has a width that is between 1-1.5 centimeters.

12. The graft of claim 8, wherein the radiopaque ink marker includes a matrix of dots having a space between adjacent dots of between 1-3 millimeters.

13. The graft of claim 8, wherein the first stent is attached to a proximal end of the tubular body and the second stent is attached to a distal end of the tubular body.

14. The graft of claim 8, wherein the radiopaque ink marker is partially opaque.

15. A method of creating a fenestration in a tubular graft in situ, the method comprising the steps of:
tracking a tubular graft to a target location within a body lumen, wherein the graft includes a first stent attached to the graft, a second stent attached to the graft, an unsupported body portion extending between the first support member and the second support member, and at least one radiopaque ink marker pattern imprinted on the unsupported body portion of the graft;
positioning the graft within the body lumen such that the radiopaque ink marker is aligned with an ostium of a side branch vessel;
radially expanding the graft;
tracking a puncture device to the radially expanded graft until a distal end of the puncture device is adjacent to the radiopaque ink marker; and
creating a fenestration in the unsupported body portion of the graft to perfuse the side branch vessel.

16. The method of claim 15, further comprising: indenting the graft with the distal end of the puncture device prior to creating a fenestration in the unsupported body portion of the graft.

17. The method of claim 15, wherein the radiopaque ink marker defines an annular band around a circumference of the tubular body.

18. The method of claim 17, wherein the annular band has a width of between 1-1.5 centimeters.

19. The method of claim 15, wherein the radiopaque ink marker includes a matrix of dots having a space between adjacent dots of between 1-3 millimeters.

20. The method of claim 15, wherein the radiopaque ink marker is partially opaque.
